(19) United States
(12) Patent Application Publication
(10) Pub. No.: US 2012/0215012 A1
(43) Pub. Date: Aug. 23, 2012

(54) PROCESS AND SYSTEM FOR PURIFICATION OF TETRAHYDROFURAN

(76) Inventors: Toshiaki Matsuo, Mito (JP); Kenichiro Oka, Mito (JP); Masayuki Kamikawa, Hitachinaka (JP); Takeyuki Kondo, Hitachi (JP); Hiroyuki Ito, Tokyo (JP); Yasunari Sase, Tokyo (JP); Chiaki Watanabe, Tokyo (JP); Tetsuji Harigai, Tokyo (JP)

(21) Appl. No.: 13/505,022

(22) PCT Filed: Oct. 30, 2009
(86) PCT No.: PCT/JP2009/068663

§ 371 Date: Apr. 30, 2012

(51) Int. Cl. C07D 307/08; B01D 3/14

(52) U.S. Cl. 549/200; 202/154

(57) ABSTRACT

A method for purifying tetrahydrofuran from a liquid containing tetrahydrofuran and as impurities at least water, 2,5-dihydrofuran and butanol, the method comprising: a first distillation step in which the liquid is subjected to distillation using a distillation column to separate into a first bottoms product containing water as a major component and a first distillate containing tetrahydrofuran, 2,5-dihydrofuran and butanol as major components, a second distillation step in which the first distillate is subjected to distillation using a distillation column to separate into a second bottoms product containing tetrahydrofuran and butanol as major components and a second distillate containing 2,5-dihydrofuran as a major component, a third distillation step in which the second bottoms product is subjected to distillation using a distillation column to separate into a third bottoms product containing butanol as a major component and a third distillate containing tetrahydrofuran as a major component, and further comprising a recirculation step in which a part of the second top liquid is recirculated into the first distillation step as a recirculation liquid and the remaining part is discharged into the outside of the system.

### FIELD OF THE INVENTION

The present invention relates to a technique for purifying tetrahydrofuran from a liquid containing tetrahydrofuran and, as impurities, at least water, 2,5-dihydrofuran and butanol.

### BACKGROUND ART

In recent years, from the viewpoint of heat resistance and mechanical strength, replacing parts conventionally made of steel materials with those of engineering plastics to reduce their weight has become a trend in the field of an automobile and the like. Among engineering plastics, polybutylene terephthalate (hereinafter PBT) is an excellent material because it has both of mechanical strength and processability and is expected to further increase in demand. PBT is synthesized by polycondensation via esterification or transesterification reaction using a glycol, 1,4-butanediol (hereinafter 1,4-BDO) and a diprotic acid, terephthalic acid (hereinafter TA) or its dimethyl ester as raw materials.

The esterification is a condensation reaction of a carboxyl group of a diprotic acid with an OH group of a glycol, which takes place at an atmospheric pressure or a slightly negative pressure, or under an inert gas atmosphere such as nitrogen to form water as a byproduct (Formula 1). Therefore, the esterification reaction is promoted by removal of water by degassing. In addition, the esterification reaction is promoted by adding a polymerization catalyst such as titanium tetrabutoxide as needed.

[Expression 1]

2HO—(CH₂)₄—OH+HOCO—C₆H₄—COOH→HO—(CH₂)₄—OCO—C₆H₄—COO—(CH₂)₄—OH+2H₂O↑ (1)

The transesterification reaction is a reaction in which the terminal glycol of one oligomer is eliminated, and a carboxyl end of the oligomer is condensed with a hydroxyl end of another oligomer to form an ester bond, wherein the oligomers are formed by the esterification reaction in the presence of a polymerization catalyst under a reduced pressure environment (Formula 2). In this case, since glycol is produced as a byproduct, the reaction is promoted by removing the glycol by degassing and the polymerization degree is increased.

[Expression 2]

HO—{(CH₂)₄—OCO—C₆H₄—CO}ₓ—O—(CH₂)₄—OH+HO—{(CH₂)₄—OCO—C₆H₄—CO}_{y}—O—(CH₂)₄—OH→HO—{(CH₂)₄—OCO—C₆H₄—CO}_{x+y}—O—(CH₂)₄—OH+HO—(CH₂)₄—OH↑ (2)

In the above esterification reaction, glycol of a raw material can undergo the dehydration reaction by catalysis of acid of the diprotic acid that is another raw material, resulting in the degradation of the glycol. Especially when glycol is ethylene glycol, diethylene glycol tends to be produced (Formula 3), and when glycol is 1,4-BDO, tetrahydrofuran (hereinafter THF) tends to be produced (Formula 4). Since these side reactions cause the decrease in the raw material yield, the side reactions are preferably suppressed as much as possible.

[Expression 3]

2HO—(CH₂)₂—OH→HO—(CH₂)₂—O—(CH₂)₂—OH+H₂O↑ (3)

HO—(CH₂)₄—OH→THF+H₂O↑ (4)

Regarding the production of PBT, a method for suppressing the production of THF in the esterification step is described in Suganuma et al., Toray Industries, Inc., J. of Soc. of Textile and Cellulose Industry Japan, Vol. 43, pp.186-191, 1987. The literature describes that the production of THF in the esterification step is promoted by an acid catalyst of TA and that the suppression of the production of THF by the reaction temperature is difficult because the activation energy (32.1 kcal) for the side reactions is equivalent to that (30.5 kcal) for the main reaction, that is, the esterification reaction. Also, it is described that although the addition of a polymerization catalyst for promoting only the main reaction is effective as a countermeasure, the production of THF per se, that is, the reduction in the raw material yield cannot be avoided.

As described above, THF is produced as a byproduct in the production of PBT. On the other hand, since THF polymerizes in the ring-opening polymerization, THF is useful as a monomer raw material for a polytetramethylene glycol (hereinafter PTMG) used as a raw material for spandex or a urethane elastomer, and if THF can be recovered, THF can be used as a valuable material. However, from the viewpoint of suppressing the coloration of PTMG, THF is required to be highly purified. Therefore, in a polymerization plant of PBT, it is important to effectively use 1,4-BDO, which is one of raw materials, by preparing a concurrent step for recovering and purifying THF.

JP Patent Publication (Kokoku) No. 6-29280 B (1994) describes a method for purifying THF in which a hydration reaction processor, a first distillation column, a hydrogenation column, a second distillation column and a third distillation column are installed in series. In the purifying method described in the disclosure, a part of 2,5-dihydrofuran (hereinafter, DHF), which is the major impurity causing the coloration of PTMG, is hydrated into hydoxyfuran using an ion-exchange resin as a catalyst by passing an aqueous solution containing THF through the hydration reaction processor, thereby reducing the vapor pressure (Formulas 5 and 6).

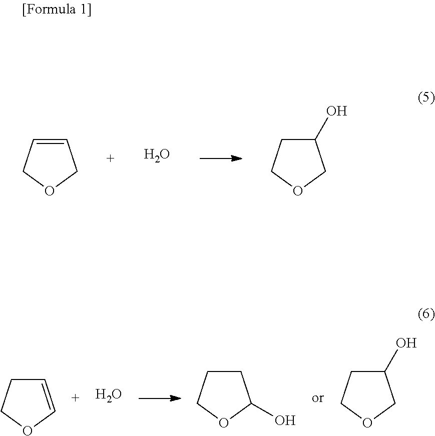

The aqueous solution passed through a hydration reaction processor is supplied into a first distillation column and is separated into a bottoms product and a distillate. When the distillate is supplied into the hydrogenation column, in the hydrogenation column, hydrogen is added to the remaining DHF in the presence of a catalyst comprising a noble metal and the DHF is converted into THF (Formula 7).

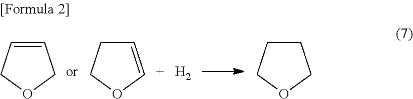

The liquid passed through the hydrogenation column is supplied into a second distillation column. In the second distillation column, THF with a large amount of moisture is a distillate and a bottoms product containing THF from which moisture is removed is supplied into a third distillation column. Here, the recovery yield of THF is increased by recirculating the distillate in the second distillation column back into the first distillation column. In the third distillation column, high purity THF is recovered as a distillate and a waste liquid containing butanol is discharged from the bottoms product. As described above, in the method described in JP Patent Publication (Kokoku) No. 6-29280 B (1994), the distillate in the second distillation column is recirculated back into the first distillation column, thereby reducing the concentration of the moisture which is difficult to remove from THF and reducing the loss of THF to increase the recovery yield of THF. Furthermore, the content of DHF in THF is reduced by adding two reaction steps of hydration and hydrogenation.

However, the addition of two reaction steps of hydration and hydrogenation may be the cause for the increase in the facility cost and the operation cost accompanied by the use of hydrogen. For this reason, the necessity existed for the development of a method for recovering and purifying THF in which the impurities of DHF and the like may be reduced in a reasonable manner in which the number of steps is small and no additional raw materials and utilities are required.

### PRIOR ART DOCUMENTS

Patent Document 1: JP Patent Publication (Kokoku) No. 06-29280

Non-Patent Document 1: Suganuma (TORAY) et al., Sen-i Gakkai-shi, Vol. 43, p. 186-191 (1987)

### SUMMARY OF THE INVENTION

### Problem to be Solved by the Invention

As described above, in the production plant of PBT, there has been desired a plant technique for economically recovering and purifying THF in order to suppress the influence of the yield reduction of raw materials due to the degradation of 1,4-BOD on economic efficiency. Therefore, an object of the present invention is to provide a method and system for purifying THF from a liquid containing THF and, as impurities, DHF and the like, in which the number of steps is small and no additional raw materials or utilities are required.

### Means to Solve the Problems

The present inventors have earnestly studied to solve the above problems. As a result, we have found that in a purifying system of THF as described in JP Patent Publication (Kokoku) No. 6-29280 B (1994) in which a first distillation column, a second distillation column and a third distillation column are installed in series, high purity THF with less impurities such as DHF can be refined with a high recovery yield without addition of a step of hydration or hydrogenation by recirculating a part of a distillate in the second distillation column back into the first distillation column and discharging the remaining part into the outside of the system, and have completed the present invention.

That is, the present invention is as follows:

(1) A method for purifying tetrahydrofuran from a liquid containing tetrahydrofuran and as impurities at least water, 2,5-dihydrofuran and butanol, the method comprising:

a first distillation step in which the liquid is subjected to distillation using a distillation column to separate into a first bottoms product containing water as a major component and a first distillate containing tetrahydrofuran, 2,5-dihydrofuran and butanol as major components,

a second distillation step in which the first distillate is subjected to distillation using a distillation column to separate into a second bottoms product containing tetrahydrofuran and butanol as major components and a second distillate containing 2,5-dihydrofuran as a major component, and

a third distillation step in which the second bottoms product is subjected to distillation using a distillation column to separate into a third bottoms product containing butanol as a major component and a third distillate containing tetrahydrofuran as a major component, and

further comprising a recirculation step in which a part of the second distillate is recirculated back into the first distillation step as a recirculation liquid and the remaining part is discharged into the outside of the system.

(2) The method for purifying tetrahydrofuran described in the above (1), in which in the recirculation step, the reflux ratio between the recirculation liquid back into the first distillation step and the discharge liquid into the outside of the system is in the range of 5:1 to 20:1.

(3) The method for purifying tetrahydrofuran described in the above (1) or (2), the method further comprising a hydrogenation process step in which 2,5-dihydrofuran contained in the first distillate is hydrogenated to be converted into tetrahydrofuran, between the first distillation step and the second distillation step.

(4) A system for purifying tetrahydrofuran, comprising:

a first distillation column comprising a raw material supply port, a discharge port of a bottoms product and a discharge port of a distillate, wherein a liquid supplied from the raw material supply port, which contains tetrahydrofuran and as impurities at least water, 2,5-dihydrofuran and butanol, is subjected to distillation to separate into a first bottoms product containing water as a major component and a first distillate containing tetrahydrofuran, 2,5-dihydrofuran and butanol as major components, the first bottoms product is discharged from the discharge port of a bottoms product, and the first distillate is discharged from the discharge port of a distillate,

a second distillation column comprising a raw material supply port, a discharge port of a bottoms product and a discharge port of a distillate, wherein the first distillate supplied from the raw material supply port is subjected to distillation to separate into a second bottoms product containing tetrahydrofuran and butanol as major components and a second distillate containing 2,5-dihydrofuran as a major component, the second bottoms product is discharged from the discharge port of a bottoms product, and the second distillate is discharged from the discharge port of a distillate,

a third distillation column comprising a raw material supply port, a discharge port of a bottoms product and a discharge port of a distillate, wherein the second bottoms product supplied from the raw material supply port is subjected to distillation to separate into a third bottoms product containing butanol as a major component and a third distillate containing tetrahydrofuran as a major component, the third bottoms product is discharged from the discharge port of a bottoms product, and the third distillate is discharged from the discharge port of a distillate,

a flow path for connecting the discharge port of the distillate in the first distillation column and the raw material supply port in the second distillation column,

a flow path for connecting the discharge port of the bottoms product in the second distillation column and the raw material supply port in the third distillation column,

a reflux pathway for connecting the discharge port of the distillate in the second distillation column and the upstream side of the first distillation column and for recirculating a part of the distillate in the second distillation column back into the first distillation column, and

a discharge path for discharging the remaining part of the distillate in the second distillation column from the discharge port of the distillate in the second distillation column into the outside of the system.

(5) The system for purifying tetrahydrofurn described in the above (4), in which it is further provided with a means for adjusting the reflux ratio between the reflux pathway and the discharge path in the range of 5:1 to 20:1.

(6) The system for purifying tetrahydrofuran described in the above (4) or (5), in which it is further provided with a hydrogenation column between the discharge port of the distillate in the first distillation column and the raw material supply port in the second distillation column.

### Effect of the Invention

The present invention can provide a method and a system of purifying THF from a liquid containing THF and, as impurities, DHF and the like, in which the number of steps is small and no additional raw materials or facilities are required.

such as a PBT or PBS (polybutylene succinate) polymerization plant. The purification method of the present invention is applied to a liquid as described above, thereby enabling to obtain high purity THF, which is a valuable material, from a liquid discharged from a polymerization plant.

### 1-2. First Distillation Step

The object of the first distillation step is that a liquid containing THF as described above is subjected to distillation using a distillation column to roughly separate and remove water which is a major impurity. In this step, a liquid containing THF as described above can be separated into a first bottoms product containing mainly of water and a first distillate containing mainly of tetrahydrofuran, 2,5-dihydrofuran and butanol.

This step can be carried out by using a distillation column comprising a raw material supply port for supplying the above liquid, a discharge port of a bottoms product for discharging a first bottoms product and a discharge port of a distillate for discharging a first distillate containing mainly of THF, DHF and butanol. The distillation column used in this step preferably has a theoretical plate number of 8 to 15 and the operation pressure is preferably one atmospheric pressure. In addition, the heating temperature of the column bottom portion is preferably 70 to 120° C.

The first bottoms product obtained in this step is supplied into a reboiler from the discharge port of the distillation column to reheat and then discharged into the outside of the system. The first bottoms product discharged into the outside of the system comprises impurities such as water and acetic acid, as major components. The reheating temperature by the reboiler is preferably 80 to 120° C. The recovery yield of THF can be further improved by returning the vapor obtained by reheating back to the distillation column.

In the column top portion of the distillation column, the distillated vapor is introduced into a condenser and the vapor is condensed to obtain a first distillate. The total amount of the first distillate obtained in this step may be used for a second distillation step described below, but it is preferable to further include a step of returning a part of the first distillate back to the distillation column in order to further improve the purity of THF. In the above step, the reflux ratio between a liquid supplied to the second distillation step and a liquid returned

### BRIEF DESCRIPTION OF THE DRAWINGS

FIG. 1 is a diagram showing an embodiment of a purification system for carrying out the whole process of the present invention.

FIG. 2 is a diagram showing another embodiment of a purification system for carrying out the whole process of the present invention.

### DETAILED DESCRIPTION OF THE PREFERRED EMBODIMENTS

### 1. Method for Purifying Tetrahydrofuran

The present invention relates to a method for purifying THF with a high purity from a liquid containing tetrahydrofuran (THF) and, as impurities, at least water, 2,5-dihydrofuran (DHF) and butanol. Each step included in the method for purifying THF of the present invention will be described below.

### 1-1. Liquid Containing Tetrahydrofuran

The method for purifying THF of the present invention may be applied to a liquid containing THF and, as impurities, at least water, DHF and butanol. In the above liquid, the concentration of THF is preferably 10 to 98%. In addition, the concentration of water is preferably 1 to 90%, the concentration of DHF is preferably 10 to 5000 ppm and the concentration of butanol is preferably 0.1 to 2%. Further, the purification method of the present invention can be similarly applied to a liquid containing, in addition to the above components, acetic acid, isopropanol, propanol, methyl ethyl ketone (MEK), 1-butylaldehyde (NBD) and the like as the other impurities. In this case, the concentration of acetic acid is preferably 0.01 to 0.5%, the concentration of isopropanol is preferably 1 to 100 ppm, the concentration of propanol is preferably 1 to 100 ppm, the concentration of MEK is preferably 1 to 50 ppm and the concentration of NBD is preferably 1 to 30 ppm.

As will be described below, in the purification method of the present invention, THF can be purified with a high purity from a liquid containing DHF and the like, as impurities. Therefore, the purification method of the present invention can be applied to a byproduct condensate discharged from the esterification step or a byproduct condensate discharged from the polycondensation step of a polymerization plant using 1,4-butanediol as a raw material back to the distillation column is preferably 1:2 to 1:4. The first distillate supplied into the second distillation step contains mainly of THF, DHF and butanol.

Water, which is a major impurity, may be efficiently removed by carrying out the first distillation step under the above conditions without reducing the recovery yield of THF.

### 1-3. Hydrogenation Process Step

The first distillate described above may be used as is for the second distillation step, but may optionally be subjected to a hydrogenation process step. The object of this step is to further improve the purity and recovery yield of THF by subjecting DHF or NBD contained as a major component of the first distillate to hydrogenation reaction.

This step may be carried out by using a hydrogenation column comprising a raw material supply port for supplying the first distillate, a hydrogen gas supply port for supplying a hydrogen gas and a discharge port for discharging a liquid after reaction. Examples of the hydrogenation column used in this step include, for example, a packed column filled with a noble metal such as ruthenium, palladium and platinum supported on graphite. If a first distillate and a hydrogen gas are supplied into such a hydrogenation column, at least a part of DHF contained in the first distillate is hydrogenated by catalytic reduction reaction to be converted into THF. Therefore, a discharge liquid of this step contains THF which is converted from DHF in addition to THF contained from the beginning in the first distillate, resulting in the increase in the total amount of THF.

If NBD is contained as an impurity, at least a part of NBD is hydrogenated by catalytic reduction reaction to be converted into butanol, which can be easily separated in a third distillation step described below. Further, when the purification method of the present invention is applied to a byproduct condensate recovered from the esterification step of the PBT polymerization plant, a sufficient purity of THF can be generally achieved without carrying out this step because the concentration of NBD contained in the condensate is not high.

The temperature inside the hydrogenation column used in this step is preferably 80 to 120° C. and the hydrogen gas partial pressure is preferably one atmospheric pressure. In addition, the residence time of the first distillate inside the column is preferably 0.25 to 1 hours.

DHF contained as an impurity can be converted into THF by carrying out a hydrogenation process step under the above conditions, thereby enabling to increase the total amount of THF and improve the recovery yield of THF. In addition, when the purification method of the present invention is applied to a liquid containing NBD as an impurity, the purity of THF can be improved by converting NBD, which is difficult to be separated, into butanol, which is easy to be separated.

### 1-4. Second Distillation Step

The object of the second distillation step is to separate and remove DHF by subjecting the first distillate or a liquid after hydrogenation reaction to distillation using the distillation column. In this step, the first distillate or the liquid after hydrogenation reaction can be separated into a second bottoms product containing THF and butanol as major components and a second distillate containing DHF as a major component.

This step can be carried out by using a distillation column comprising a raw material supply port for supplying the first distillate, a discharge port of a bottoms product for discharging the second bottoms product containing THF and butanol as major components and a discharge port of a distillate for discharging the second distillate containing DHF as a major component. The distillation column used in this step preferably has a theoretical plate number of 12 to 16 and the operation pressure is preferably 8 to 9 atmospheric pressure. In addition, the heating temperature of the column bottom portion is preferably 120 to 180° C.

The second bottoms product obtained in this step is supplied into a reboiler from the discharge port of the distillation column to reheat and then supplied into a third distillation step as the second bottoms product. The second bottoms product supplied into the third distillation step contains THF and butanol as major components. The reheating temperature by the reboiler is preferably 120 to 180° C. DHF, which is an impurity, can be further separated and removed by returning the vapor obtained by reheating back to the distillation column.

In the column top portion of the above distillation column, the distillated vapor is introduced into a condenser and the vapor is condensed to obtain a second distillate. The total amount of the second distillate obtained in this step may be used for a recirculation step described below, but it is preferable to further include a step of returning a part of the second distillate back to the distillation column in order to further improve the recovery yield of THF. In the above step, the reflux ratio between a liquid supplied to the recirculation step and a liquid returned back to the distillation column is preferably 1:0.1 to 1:0.6. The second distillate supplied into the recirculation step contains DHF as a major component and also contains water forming an azeotrope with DHF.

DHF, which is an impurity, may be effectively removed by carrying out the second distillation step under the above conditions without reducing the recovery yield of THF.

### 1-5. Recirculation Step

The object of the recirculation step is to further improve the efficiency of separation and removal of DHF and water, which are impurities contained in the distillate, by recirculating a part of the second distillate separated in the second distillation step as a recirculation liquid back into the first distillation step and discharging the remaining part into the outside of the system.

Since THF, water and DHF form an azeotrope, it is generally difficult to separate these compounds by distillation. As a method for separating and removing water from a liquid containing THF, water and DHF, for example, JP Patent Publication (Kokoku) No. 6-29280 B (1994) describes a method for purifying THF which comprises a hydration reaction processor, a first distillation column, a hydrogenation column, a second distillation column and a third distillation column in series. According to the literature, the moisture content into the bottoms product may be reduced by carrying out the distillation under a pressure higher than atmospheric pressure in the second distillation column, and water may be further separated and removed by recirculating the distillate back into the first distillation column. On the other hand, in the method described in JP Patent Publication (Kokoku) No. 6-29280 B (1994), DHF is planned to be removed by the hydrogenation reaction in the hydrogenation column and DHF is not assumed to be contained in a liquid supplied into the second distillation column. Therefore, the separation and removal of DHF is not described in JP Patent Publication (Kokoku) No. 6-29280 B (1994).

However, the present inventors have found that DHF can be completely removed from a second bottoms product and transferred into a second distillate by carrying out the recirculation step described in this section, even if the hydrogenation process step is not carried out as an essential step. That is, water contained in a second distillate after recirculation is removed as a component of a first bottoms product after recirculation by recirculating a part of the second distillate as a recirculation liquid back into the first distillation step, whereas DHF is supplied again into the second distillation step as a component of the first distillate after recirculation. Therefore, DHF is concentrated into the second distillate by carrying out the recirculation step.

In addition, the present inventors have found that DHF can be separated and removed by recirculating a part of the second distillate back into the first distillation step as a recirculation liquid and discharging the remaining part into the outside of the system as a discharge liquid, instead of recirculating the total amount of the second distillate back into the first distillation step as described in JP Patent Publication (Kokoku) No. 6-29280 B (1994). The second distillate also contains THF, which should be separated as a major component of the second bottoms product. For this reason, in the recirculation step, if the flow rate of the discharge liquid discharged into the outside of the system is increased, the separation efficiency of DHF is improved, but the amount of THF discharged simultaneously is increased. As a result, the recovery yield of THF is reduced and the economic efficiency of the purification method of the present invention is deteriorated. On the other hand, if the flow rate of the recirculation liquid back to the first distillation step is increased, the recovery yield of THF is improved, but the amount of DHF supplied again into the second distillation step through the first distillation step is increased. As a result, the amount of DHF contained in the second bottoms product is increased, thereby reducing the purity of THF obtained in a third distillation step described below.

As described above, the reflux ratio between the recirculation liquid back into the first distillation step and the discharge liquid into the outside of the system is an important factor defining the recovery yield and purity of THF in the purification method of the present invention. Therefore, in this step, the reflux ratio between the recirculation liquid back into the first distillation step and the discharge liquid into the outside of the system is preferably in the range of 5:1 to 20:1. The above reflux ratio can be adjusted by optionally setting the flow rate of the recirculation liquid back into the first distillation step and the discharge liquid into the outside of the system by combination of flow rate measuring means such as a flow rate meter or a current meter and flow rate adjusting means such as a flow rate adjusting valve.

The recovery yield of THF can be improved by carrying out the recirculation step under the above conditions and the purity of THF can be improved by removing DHF and water, which are impurities.

### 1-6. Third Distillation Step

The object of the third distillation step is to separate THF, which is an objective product, by subjecting the second bottoms product to distillation using a distillation column. In this step, the second bottoms product can be separated into a third bottoms product containing impurities such as butanol, acetic acid, isopropanol, propanol, methyl ethyl ketone (MEK) and 1-butylaldehyde (NBD) as major components and a third distillate containing THF as a major component.

This step can be carried out by using a distillation column comprising a raw material supply port of a bottoms product for supplying the second bottoms product, a discharge port of a bottoms product for discharging the third bottoms product containing impurities such as butanol as a major component, and a discharge port of a distillate for discharging the third distillate containing THF as a major component, which is an objective product. The distillation column used in this step preferably has a theoretical plate number of 15 to 25 and the operation pressure is preferably one atmospheric pressure. In addition, the heating temperature of the column bottom portion is preferably 60 to 90° C.

The third bottoms product obtained in this step is supplied into a reboiler from the discharge port of the distillation column to reheat and then discharged into the outside of the system as the third bottoms product. The third bottoms product discharged into the outside of the system contains impurities such as butanol, acetic acid, isopropanol, propanol, MEK and NBD as major components. The reheating temperature by the reboiler is preferably 60 to 75° C. The recovery yield of THF, which is an objective product, can be improved by returning the vapor obtained by reheating back to the distillation column.

In the column top portion of the above distillation column, the distillated vapor is introduced into a condenser and the vapor is condensed to obtain a third distillate. The total amount of the third distillate obtained in this step may be discharged as is into the outside of the system as high purity THF, but it is preferable to further include a step of returning a part of the third distillate back to the distillation column in order to further improve the purity of THF. In the above step, the reflux ratio between a liquid discharged into the outside of the system and a liquid returned back to the distillation column is preferably 1:0.3 to 1:1. The liquid discharged into the outside of the system is high purity of THF, which is an objective product.

High purity of THF, which is an objective product, can be obtained by carrying out the third distillation step under the above conditions without reducing the recovery yield and purity of THF.

As described above, in the purification method of the present invention, THF, which is an objective product, can be obtained from a liquid containing THF and, as impurities, at least water, DHF and butanol without reducing the recovery yield and purity of THF. Therefore, high purity of THF can be obtained by the purification method of the present invention with small number of steps.

### 2. System for Purifying Tetrahydrofuran

Further, the present invention relates to a system for purifying THF with high purity from a liquid containing THF and, as impurities, at least water, DHF and butanol. The system for purifying THF of the present invention will be described below based on the accompanying drawings, but the purification system of the present invention is not limited thereto.

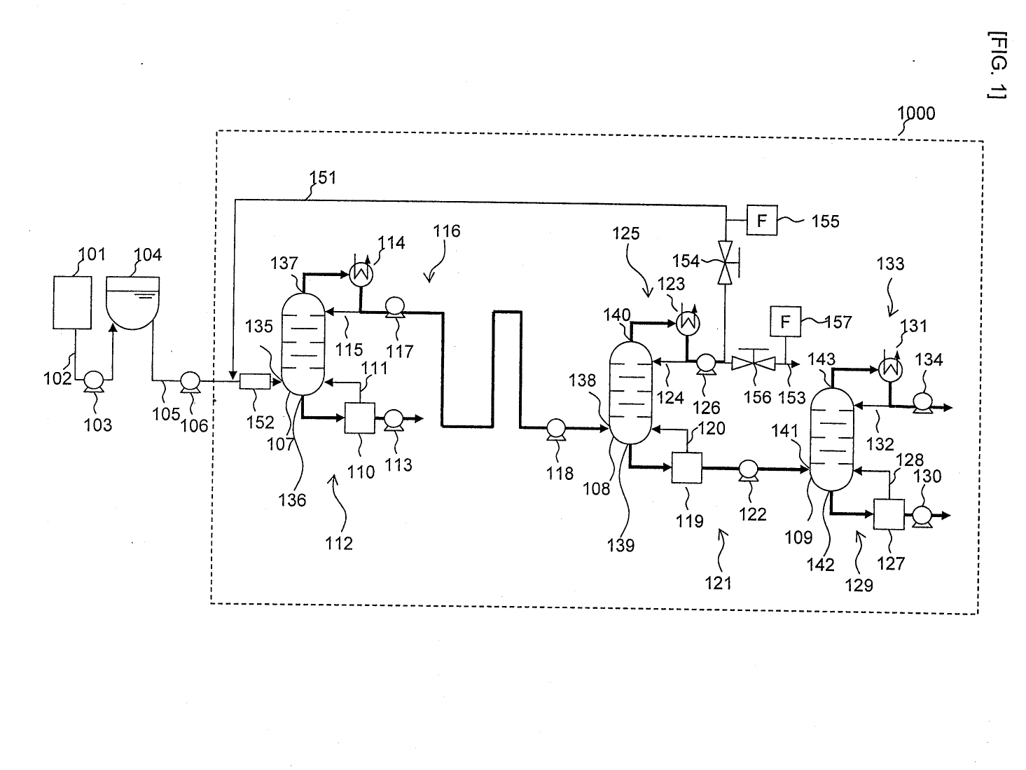

A basic embodiment of a system for purifying THF of the present invention shown in FIG. 1, the system mainly comprising:

a first distillation column 107 comprising a raw material supply port 135, a discharge port 136 of a bottoms product and a discharge port 137 of a distillate, wherein a liquid supplied from the raw material supply port 135, which contains THF and, as impurities, at least water, DHF and butanol, is subjected to distillation to separate into a first bottoms product containing water as a major component and a first distillate containing THF, DHF and butanol as major components, the first bottoms product is discharged from discharge port 136 of a bottoms product and the first distillate is discharged from the discharge port 137 of a distillate,

a second distillation column 108 comprising a raw material supply port 138, a discharge port 139 of a bottoms product and a discharge port 140 of a distillate, wherein the first distillate supplied from the raw material supply port 138 is subjected to distillation to separate into a second bottoms product containing THF and butanol as major components and a second distillate containing DHF as a major component, the second bottoms product is discharged from the discharge port 139 of a bottoms product and the second distillate is discharged from the discharge port 140 of a distillate,

a third distillation column 109 comprising a raw material supply port 141, a discharge port 142 of a bottoms product and a discharge port 143 of a distillate, wherein the second bottoms product supplied from the raw material supply port 141 is subjected to distillation to separate into a third bottoms product containing butanol as a major component and a third distillate containing THF as a major component, the third bottoms product is discharged from the discharge port 142 of a bottoms product and the third distillate is discharged from the discharge port 143 of a distillate,

a flow path 116 for connecting the discharge port 137 of a distillate in the first distillation column 107 and the raw material supply port 138 in the second distillation column 108,

a flow path 121 for connecting the discharge port 139 of a bottoms product in the second distillation column 108 and the raw material supply port 141 in the third distillation column 109,

a reflux pathway 151 for connecting the discharge port 140 of a distillate in the second distillation column 108 and the upstream side of the first distillation column 107 and for recirculating a part of the distillate in the second distillation column 108 back into the first distillation column 107, and

a discharge path 153 for discharging the remaining part of the distillate in the second distillation column 108 from the discharge port 140 of a distillate in the second distillation column 108 into the outside of the system.

Here, each of the flow path and discharge path is not always formed by a single flow path member, and a plurality of flow path members may be disposed in serial or parallel to form a flow path, and further other units such as a transfer pump, a reboiler, a condenser and a hydrogenation column may intervene in the course of the flow path. As the above flow path members, an optional member such as a pipe may be used.

In the purification system of the present invention, a liquid supplied from the raw material supply port 135 in the first distillation column 107 preferably has the same composition as the liquid described in above 1-1. Such a liquid is generally discharged as a byproduct condensate from the esterification step or polycondensation step of the PBT polymerization plant. Therefore, the purification system of the present invention is preferably disposed on the downstream side of a plant 101 for polyester in which 1,4-butanediol of the PBT or PBS polymerization plant and the like is used as a raw material. In case of the above, a storage tank 104 is disposed on the downstream side of the plant 101 and a liquid discharged from the plant 101 is supplied into the storage tank 104 through a flow path 102. A transport system such as a transfer pump 103 may be disposed in the flow path 102 where necessary.

A liquid containing THF and, as impurities, at least water, DHF and butanol is supplied from the storage tank 104 through a flow path 105 into the raw material supply port 135 in the first distillation column 107. A transport system such as a transfer pump 106 may be disposed in the flow path 105 where necessary. In the first distillation column 107, the liquid is separated into a first bottoms product containing water as a major component and a first distillate containing THF, DHF and butanol as major components. The first distillation column 107 preferably has a theoretical plate number of 8 to 15 and the operation pressure is preferably one atmospheric pressure. In addition, the heating temperature of the column bottom portion is preferably 90 to 120° C.

A first bottoms product discharged from the discharge port 136 in the first distillation column 107 is supplied into a reboiler 110. In the reboiler 110, the vapor which is generated by reheating the first bottoms product is returned back to the first distillation column 107 through a flow path 111 and the first bottoms product after reheating is discharged into the outside of the system through a discharge path 112. In this case, the discharge path 112 is configured by the flow path members from the discharge port 136 to the reboiler 110, the reboiler 110 and the flow path members from the reboiler 110 to the outside of the system. In addition, a transport system such as a transfer pump 113 may be disposed in the discharge path 112 where necessary. The reheating temperature by the reboiler 110 is preferably 80 to 120° C. The first bottoms product discharged contains water as a major component. The recovery yield of THF can be further improved by returning the vapor obtained by reheating back to the first distillation column 107.

In the column top portion of the first distillation column 107, the vapor distillated from the discharge port 137 is introduced into a condenser 114 and the vapor is condensed to obtain a first distillate. The total amount of the first distillate obtained in this step may be supplied into the second distillation column 108 through the flow path 116, but a part of the first distillate is preferably returned back to the first distillation column 107 through the flow path 115 in order to further improve the purity of THF. In case of the above, the reflux ratio between a liquid supplied into the second distillation column 108 through the flow path 116 and a liquid returned back to the distillation column through the flow path 115 is preferably 1:2 to 1:4. In this case, the flow path 116 is configured by the flow path members from the discharge port 137 to the condenser 114, the condenser 114 and the flow path members from the condenser 114 to the raw material supply port 138 in the second distillation column 108. In addition, a transport system such as a transfer pump 117 may be disposed in the flow path 116 where necessary. A first distillate supplied into the second distillation column 108 through the flow path 116 contains THF, DHF and butanol as major components.

The first distillate is supplied into the raw material supply port 138 in the second distillation column 108 through the flow path 116. In the second distillation column 108, the first distillate is separated into a second bottoms product containing THF and butanol as major components and a second distillate containing DHF as a major component. The second distillation column 108 preferably has a theoretical plate number of 12 to 16 and the operation pressure is preferably 8 to 9 atmospheric pressure. In addition, the heating temperature of the column bottom portion is preferably 130 to 170° C.

A second bottoms product discharged from the discharge port 139 in the second distillation column 108 is supplied into a reboiler 119. In the reboiler 119, the vapor which is generated by reheating the second bottoms product is returned back to the second distillation column 108 through a flow path 120 and the second bottoms product after reheating is supplied into the third distillation column 109 through the flow path 121. In this case, the flow path 121 is configured by the flow path members from the discharge port 139 to the reboiler 119, the reboiler 119 and the flow path members from the reboiler 119 to the raw material supply port 141 in the third distillation column 109. In addition, a transport system such as a transfer pump 122 may be disposed in the flow path 121 where necessary. The reheating temperature by the reboiler 119 is preferably 120 to 180° C. The second bottoms product supplied into the third distillation column 109 through the flow path 121 contains THF and butanol as major components. Water and DHF, which are impurities, can be further separated and removed by returning the vapor obtained by reheating back to the second distillation column 108.

In the column top portion of the second distillation column 108, the vapor distillated from the discharge port 140 is introduced into a condenser 123 and the vapor is condensed to obtain a second distillate. The total amount of the second distillate obtained in this step may be supplied into the reflux pathway 151 and the discharge path 153, but a part of the second distillate is preferably returned back to the second distillation column 108 through the flow path 124 in order to further improve the recovery yield of THF. In case of the above, the reflux ratio between a liquid supplied into the reflux pathway 151 and the discharge path 153 and a liquid returned back to the distillation column through the flow path 124 is preferably 1:0.1 to 1:0.6. The second distillate supplied into the reflux pathway 151 and the discharge path 153 contains DHF as a major component. The second distillate may be directly supplied from the discharge port of the second distillate into the reflux pathway and the discharge path, and as shown in FIG. 1, may be supplied from the discharge port 140 of the second distillate into the reflux pathway 151 and the discharge path 153 through a flow path 125 which is connected to the discharge port 140 of the second distillate at one end and branched into the reflux pathway 151 and the discharge path 153 at another end. In this case, the flow path 125 is configured by the flow path members from the discharge port 140 to the condenser 123, the condenser 123 and the flow path members from the condenser 123 to the branched point into the reflux pathway 151 and the discharge path 153. In addition, a transport system such as a transfer pump 126 may be disposed in the flow path 125 where necessary.

The second distillate contains DHF as a major component and further contains water forming an azeotrope with DHF. As described above, a part of the second distillate is supplied into the raw material supply port 135 in the first distillation column 107 through the reflux pathway 151. A liquid supplied through the reflux pathway 151 and a liquid supplied through the flow path 105 are mixed by a mixing means such as a static mixer 152 where necessary and the mixture may be supplied into the raw material supply port 135 in the first distillation column 107. The second distillate containing DHF as a major component, which is supplied through the reflux pathway 151, is again subjected to distillation in the first distillation column 107. The first distillate after recirculation, which contains DHF as a major component, is again supplied into the raw material supply port 138 in the second distillation column 108 through the flow path 116, and the first bottoms product after recirculation, which contains water as a major component, is discharged into the outside of the system through the discharge path 112.

On the other hand, the remaining part of the second distillate is discharged into the outside of the system through the discharge path 153, thereby enabling to reduce the amount of DHF contained in the liquid supplied through the reflux pathway 151, and as a result, enabling to reduce the amount of DHF contained in the bottoms product in the second distillation column 108. Therefore, the purity of THF obtained in the third distillation column 109 described below can be improved.

As described above, in the purification system of the present invention, the recovery yield and purity of THF can be improved by adjusting the flow rate of the reflux pathway 151 and the discharge path 153. Therefore, a means for adjusting the reflux ratio between the reflux pathway 151 and the discharge path 153 is preferably disposed on the downstream side of the second distillation column 108. Example of such a means include, for example, a combination of a flow rate measuring means such as flowmeters 155 and 157 and a flow rate adjusting means such as flow rate adjusting valves 154 and 156. The flow rate measuring means and flow rate adjusting means may be disposed as a separate part as shown in FIG. 1, or may be disposed as a single part having both functions. The above flow rate measuring means and flow rate adjusting means may be disposed on the downstream side of the second distillation column 108, may be disposed on each of the reflux pathway 151 and the discharge path 153 as shown in FIG. 1, or may be disposed on either one of the reflux pathway 151 and the discharge path 153.

The second bottoms product is supplied into the raw material supply port 141 in the third distillation column 109 through the flow path 121. In the third distillation column 109, the second bottoms product is separated into a third bottoms product containing butanol as a major component and a third distillate containing THF as a major component. The third distillation column 109 preferably has a theoretical plate number of 15 to 25 and the operation pressure is preferably one atmospheric pressure. In addition, the heating temperature of the column bottom portion is preferably 60 to 90° C.

The third bottoms product discharged from the discharge port 142 in the third distillation column 109 is supplied into a reboiler 127. In the reboiler 127, the vapor generated by reheating the third bottoms product is returned back to the third distillation column 109 through a flow path 128 and the third bottoms product after reheating is discharged into the outside of the system through a discharge path 129. In this case, the discharge path 129 is configured by the flow path members from the discharge port 142 to the reboiler 127, the reboiler 127 and the flow path members from the reboiler 127 to the outside of the system. In addition, a transport system such as a transfer pump 130 may be disposed in the discharge path 129 where necessary. The reheating temperature by the reboiler 127 is preferably 60 to 75° C. The bottoms product discharged into the outside of the system through the discharge path 129 contains butanol as a major component. The recovery yield of THF, which is an objective product, can be improved by returning the vapor obtained by reheating back to the third distillation column 109.

In the column top portion of the third distillation column 109, the vapor distillated from the discharge port 143 is introduced into a condenser 131 and the vapor is condensed to obtain a third distillate. The total amount of the third distillate obtained in this step may be supplied into a discharge path 133, but a part of the third distillate is preferably returned back to the third distillation column 109 through a flow path 132 in order to further improve the purity of THF. In case of the above, the reflux ratio between a liquid supplied into the discharge path 133 and a liquid returned back to the distillation column through the flow path 132 is preferably 1:0.3 to 1:1. In this case, the discharge path 133 is configured by the flow path members from the discharge port 143 to the condenser 131, the condenser 131 and the flow path members from the condenser 131 to the outside of the system. In addition, a transport system such as a transfer pump 134 may be disposed in the discharge path 133 where necessary. A third distillate discharged from the discharge path 133 is high purity of THF, which is an objective product.

In the purification system of the embodiment shown in FIG. 1, THF, which is an objective product, can be obtained from a liquid containing THF and, as impurities, at least water, DHF and butanol, without reducing the recovery yield and purity of THF. Since the purification system has a small number of steps and requires no additional raw materials and facilities, high purity of THF can be obtained without increasing the production cost and the facility cost.

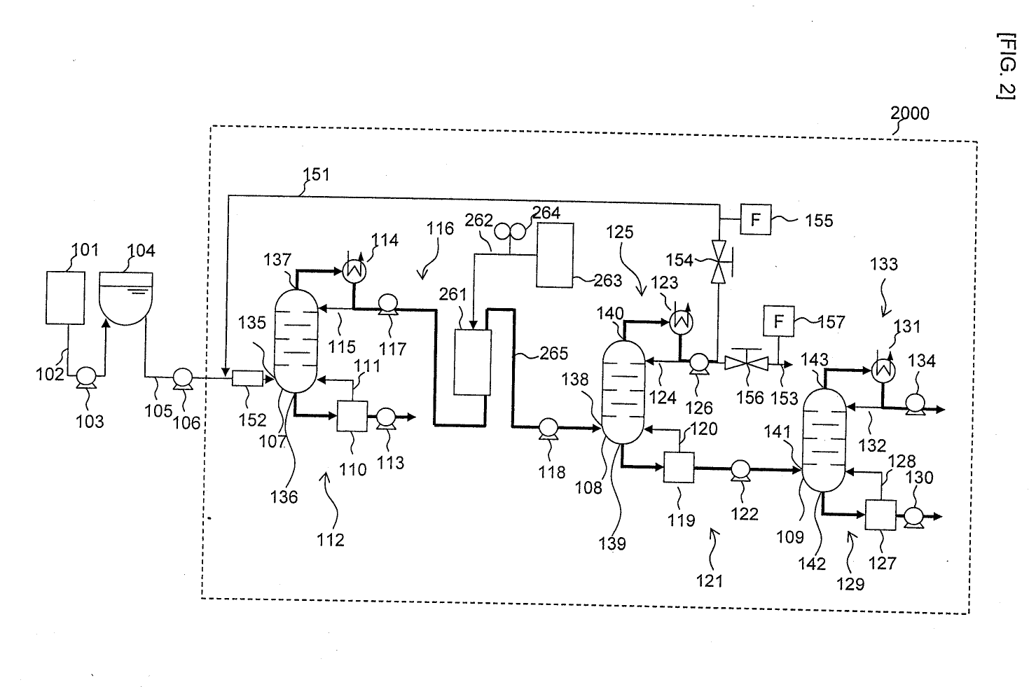

Further, another embodiment of the purification system of the present invention is shown in FIG. 2. The embodiment of FIG. 2 has the same configuration as the purification system shown in FIG. 1 except that a hydrogenation column 261 is disposed between a discharge port of a distillate in a first distillation column and a raw material supply port in a second distillation column. In the embodiment of FIG. 2, there is disposed the hydrogenation column 261 comprising a raw material supply port, a hydrogen gas supply port for supplying a hydrogen gas and a discharge port for discharging a liquid after reaction in the course of a flow path for connecting the discharge port of a distillate in a first distillation column and the raw material supply port in a second distillation column. In this case, the first distillate containing THF, DHF and butanol as major components is supplied into the raw material supply port in the hydrogenation column 261 through the flow path members from the discharge port 137 to the condenser 114, the condenser 114 and the flow path members from the condenser 114 to the raw material supply port in the hydrogenation column 261. The hydrogenation column 261 is preferably, for example, a packed column filled with a noble metal such as ruthenium, palladium and platinum supported on graphite. The hydrogen gas supply port in the hydrogenation column 261 is connected to a tank 263 filled with a hydrogen gas through a pipe 262, and the hydrogen gas is supplied from the tank 263 into the hydrogenation column 261. The hydrogen gas partial pressure supplied is adjusted at a suitable partial pressure by a flow rate adjusting means such as a regulator 264 disposed between the hydrogenation column 261 and the tank 263. In addition, the hydrogenation column 261 comprises a heater and the inside of the hydrogenation column is kept at a suitable temperature. As a result, in the inside of the hydrogenation column 261, at least a part of DHF contained in the distillate in the first distillation column 107 is hydrogenated by catalytic reduction reaction to be converted into THF. If NBD is contained as an impurity, at least a part of NBD is hydrogenated by catalytic reduction reaction and converted into butanol, which is easily separated in the third distillation column 109.

The temperature of the inside of the hydrogenation column 261 is preferably 80 to 120° C., and the hydrogen gas partial pressure is preferably one atmospheric pressure. In addition, the residence time of the distillate in the first distillation column 107 is preferably 0.25 to 1 hours.

In the purification system of the embodiment shown in FIG. 2, the total amount of THF is increased by converting DHF contained as an impurity into THF, resulting in the improvement in the recovery yield of THF. In addition, when the purification system is applied to a liquid containing NBD as an impurity, the purity of THF can be improved by converting NBD, which is difficult to be separated, into butanol which is easy to be separated.

Hereinafter, the present invention will be described in more detail based on an example, but the scope of the present invention is not limited to the example.

### EXAMPLES

### Example

The system for purifying THF used in the present example comprises: disposing in series a first distillation column comprising a raw material supply port, a discharge port of a bottoms product and a discharge port of a distillate, wherein a byproduct condensate of a PBT polymerization plant, which contains THF, DHF and butanol supplied from the raw material supply port, is subjected to distillation to separate into a first bottoms product containing mainly of water and a first distillate containing mainly of THF, DHF and butanol, the first bottoms product is discharged from the discharge port of a bottoms product and the first distillate is discharged from the discharge port of a distillate, a second distillation column comprising a raw material supply port, a discharge port of a bottoms product and a discharge port of a distillate, wherein the first distillate supplied from the raw material supply port is subjected to distillation to separate into a second bottoms product containing mainly of THF and butanol and a second distillate containing mainly of DHF, the second bottoms product is discharged from the discharge port of a bottoms product and the second distillate is discharged from the discharge port of a distillate, and a third distillation column comprising a raw material supply port, a discharge port of a bottoms product and a discharge port of a distillate, wherein the second bottoms product supplied from the raw material supply port is subjected to distillation to separate into a third bottoms product containing mainly of butanol and a third distillate containing mainly of THF, the third bottoms product is discharged from the discharge port of a bottoms product and the third distillate is discharged from the discharge port of a distillate; and further comprises a reflux pathway for connecting the discharge port of the distillate in the second distillation column and the upstream side of the first distillation column and for recirculating a part of the distillate in the second distillation column back into the first distillation column and a discharge path for discharging the remaining part of the distillate in the second distillation column from the discharge port of the distillate in the second distillation column into the outside of the system. Hereinafter, the example of the present invention will be explained based of FIG. 2.

A byproduct condensate was supplied from the PBT polymerization plant 101 through the flow path 102 into the byproduct condensate tank 104 using the transfer pump 103. The condensate contained 68% of water, 30.83% of THF, 0.82% of butanol, 0.22% of acetic acid, 1200 ppm of DHF, 35 ppm of isopropanol, 10 ppm of NBD and 10 ppm of MEK. Assuming that in the byproduct condensate tank 104, 1,4-BDO is contained in the condensate, the byproduct condensate tank 104 is kept at a temperature (20 to 25° C.) higher than or equal to the melting point of 1,4-BDO as needed.

Into the first distillation column 107 was supplied 1995 parts of the byproduct condensate from the byproduct condensate tank 104 through the flow path 105 using the transfer pump 106. In addition, into the first distillation column 107 was recirculated 298 parts of a distillate in the second distillation column 108 described below from the second distillation column 108 through the reflux pathway 151. The recirculation liquid and the byproduct condensate were mixed using the static mixer 152 and then the liquid mixture was supplied into the first distillation column 107. The first distillation column 107 had a theoretical plate number of 15, the column bottom temperature was set at 100° C., the operation pressure was set at one atmospheric pressure, and the recirculation ratio of the distillate was set at 2.9. The bottoms product in the first distillation column 107 contained 99.06% of water, 0.56% of butanol, 0.38% of acetic acid and 0.01% of THF as high-boiling components, and 1368 parts of the bottoms product was discharged into the outside of the system through the discharge path 112 using a transfer pump 113. On the other hand, the distillate in the first distillation column 107 contained 94.5% of THF, 2.32% of DHF, 0.92% of butanol, 0.01% of isopropanol, 20 ppm of NBD and 20 ppm of MEK, and 925 parts of the distillate was supplied into the hydrogenation column 261 through the flow path 116 consisting of the flow path members from the discharge port 137 to the condenser 114, the condenser 114 and the flow path members from the condenser 114 to the raw material supply port in the hydrogenation column 261 using the transfer pump 117.

The hydrogenation column 261 is a packed column filled with a pellet catalyst obtained by supporting 2% of metal ruthenium on graphite, the column bottom temperature was set at 100° C., the operation pressure was set at 9.5 atmospheric pressure and the residence time was set at 0.5 hours. The operation pressure was adjusted to the above pressure by the regulator 264 and a hydrogen gas was supplied from the tank 263 through the pipe 262 into the hydrogenation column 261. A liquid supplied into the hydrogenation column 261 was brought into contact with the hydrogen gas in the column, thereby reducing a part of DHF to THF. Into the second distillation column 108 was supplied 925 parts of the hydrogenation reaction liquid through the flow path members 265 from the discharge port in the hydrogenation column 261 to the raw material supply port 138 in the second distillation column 108 using the transfer pump 118. In addition, the hydrogenation column 261 may be omitted where necessary. Hereinafter, a case will be described where the hydrogenation column 261 is omitted.

The second distillation column 108 had a theoretical plate number of 14, the column bottom temperature was set at 150° C., the operation pressure was set at 8.4 atmospheric pressure, and the recirculation ratio of the distillate was set at 0.3. The bottoms product in the second distillation column 108 contained 98.54% of THF, 1.41% of butanol, 0.04% of DHF, 0.01% of isopropanol, 0.01% of acetic acid, 50 ppm of water and 10 ppm of MEK as high-boiling components, and 601 parts of the bottoms product was supplied into the third distillation column 108 through the flow path 121 consisting of the flow path members from the discharge port 139 to the reboiler 119, the reboiler 119 and the flow path members from the reboiler 119 to the raw material supply port 141 in the third distillation column 108 using the transfer pump 122. On the other hand, the distillate in the second distillation column 108 contained 87% of THF, 6.42% of water, 5.55% of DHF, 10 ppm of NBD and 10 ppm of MEK and was discharged from the second distillation column 108 through the flow path 125 using the transfer pump 126. The flow path 125 consists of the flow path members from the discharge port 140 to the condenser 123, the condenser 123 and the flow path members from the condenser 123 to the branched point into the reflux pathway 151 and the discharge path 153. The distillate discharged was flowed into the reflux pathway 151 and the discharge path 153 through the flow path 125. The flow rate adjusting valves 154 and 156 and the flowmeters 155 and 157 are installed on the reflux pathway 151 and the discharge path 153, and the reflux ratio was adjusted so that 298 parts of the liquid is flowed in the reflux pathway 151 and 26 parts of the liquid is flowed in the discharge path 153 by operating the flow rate adjusting valves 154 and 156. The liquid flowing in the reflux pathway 151 was recirculated back into the first distillation column 107. In addition, the liquid flowing in the discharge path 153 was discharged into the outside of the system as a discharge liquid. Therefore, the reflux ratio between the recirculation liquid and the discharge liquid into the outside of the system at this time was 11.46.

The third distillation column 109 had a theoretical plate number of 19, the column bottom temperature was set at 67° C., the operation pressure was set at one atmospheric pressure, and the recirculation ratio of the distillate was set at 0.6. The bottoms product in the third distillation column 109 contained 97.85% of butanol, 0.8% of THF, 0.64% of isopropanol, 0.36% of acetic acid, 0.02% of water, 0.11% of MEK and 0.11% of NBD as high-boiling components, 9 parts of the bottoms product was discharged into the outside of the system through the discharge path 129 consisting of the flow path members from the discharge port 142 to the reboiler 127, the reboiler 127 and the flow path members from the reboiler 127 to the outside of the system using the transfer pump 130. On the other hand, the distillate in the third distillation column contained 99.96% of THF and 0.04% of DHF, and 593 parts of the distillate was discharged through the discharge path 133 comprised of the flow path members from the discharge port 143 to the condenser 131, the condenser 131 and the flow path members from the condenser 131 to the outside of the system using the transfer pump 134. The distillate in the third distillation column 109 is high purity of THF, which is a final product in the present example.

The purity of THF recovered by the present example was 99.96% and the recovery yield of THF was 96.4%, which achieved a targeted purity of 99.9% or more and a targeted recovery yield of 90% or more.

### Comparative Example

The purification system of THF of the present invention described in the above example was not applied, and a purification system of the distillate in the second distillation column was operated without discharging a part of the distillate into the outside of the system. As a result, it was found that a THF solution of the final product contained 7% of DHF and a significant deterioration in purity occurred.

### INDUSTRIAL APPLICABILITY

As described above, according to the purification system of THF of the present invention, high purity of THF can be purified from a byproduct condensate containing THF generated in a PBT polymerization plant with small numbers of steps and reasonable facilities as well as with a high recovery yield.

All of the publications, patents and patent applications cited in this description are herein incorporated by reference in their entirety.

### Description of Symbols

107 . . . First distillation column

108 . . . Second distillation column

109 . . . Third distillation column

135 . . . Raw material supply port in first distillation column

136 . . . Discharge port of bottoms product in first distillation column

137 . . . Discharge port of distillate in first distillation column

138 . . . Raw material supply port in second distillation column

139 . . . Discharge port of bottoms product in second distillation column

140 . . . Discharge port of distillate in second distillation column

141 . . . Raw material supply port in third distillation column

142 . . . Discharge port of bottoms product in third distillation column

143 . . . Discharge port of distillate in third distillation column

151 . . . Reflux pathway

116, 121, 125 . . . Flow path

112, 129, 133, 153 . . . Discharge path

154, 156 . . . Flow rate adjusting valve

155, 157 . . . Flowmeter

261 . . . Hydrogenation column

262 . . . Pipe

263 . . . Tank

264 . . . Regulator

1000 . . . Purification system of tetrahydrofuran

2000 . . . Purification system of tetrahydrofuran

1. A method for purifying tetrahydrofuran from a liquid comprising tetrahydrofuran and as impurities at least water, 2,5-dihydrofuran and butanol, the method comprising:
a first distillation step in which the liquid is subjected to distillation using a distillation column to separate into a first bottoms product containing water as a major component, and a first distillate containing tetrahydrofuran, 2,5-dihydrofuran and butanol as major components,
a second distillation step in which the first distillate is subjected to distillation using a distillation column to separate into a second bottoms product containing tetrahydrofuran and butanol as major components and a second distillate containing 2,5-dihydrofuran as a major component, and
a third distillation step in which the second bottoms product is subjected to distillation using a distillation column to separate into a third bottoms product containing butanol as a major component and a third distillate containing tetrahydrofuran as a major component, and
further comprising a recirculation step in which a part of the second distillate is recirculated back into the first distillation step as a recirculation liquid and the remaining part is discharged into the outside of the system,
wherein in the recirculation step, the reflux ratio between the recirculation liquid back into the first distillation step and the discharge liquid into the outside of the system is in the range of 5:1 to 20:1.

2. (canceled)

3. The method for purifying tetrahydrofuran according to claim 1, further comprising a hydrogenation process step in which 2,5-dihydrofuran contained in the first distillate is hydrogenated to be converted into tetrahydrofuran, between the first distillation step and the second distillation step.

4. A system for purifying tetrahydrofuran, comprising:
a first distillation column comprising a raw material supply port, a discharge port of a bottoms product and a discharge port of a distillate, wherein a liquid supplied from the raw material supply port, which contains tetrahydrofuran and as impurities at least water, 2,5-dihydrofuran and butanol, is subjected to distillation to separate into a first bottoms product containing water as a major component and a first distillate containing tetrahydrofuran, 2,5-dihydrofuran and butanol as major components, the first bottoms product is discharged from the discharge port of a bottoms product, and the first distillate is discharged from the discharge port of a distillate,
a second distillation column comprising a raw material supply port, a discharge port of a bottoms product and a discharge port of a distillate, wherein the first distillate supplied from the raw material supply port is subjected to distillation to separate into a second bottoms product containing tetrahydrofuran and butanol as major components and a second distillate containing 2,5-dihydrofuran as a major component, the second bottoms product is discharged from the discharge port of a bottoms product, and the second distillate is discharged from the discharge port of a distillate,
a third distillation column comprising a raw material supply port, a discharge port of a bottoms product and a discharge port of a distillate, wherein the second bottoms product supplied from the raw material supply port is subjected to distillation to separate into a third bottoms product containing butanol as a major component and a third distillate containing tetrahydrofuran as a major component, the third bottoms product is discharged from the discharge port of a bottoms product and the third distillate is discharged from the discharge port of a distillate,
a flow path for connecting the discharge port of the distillate in the first distillation column and the raw material supply port in the second distillation column,
a flow path for connecting the discharge port of the bottoms product in the second distillation column and the raw material supply port in the third distillation column,
a reflux pathway for connecting the discharge port of the distillate in the second distillation column and the upstream side of the first distillation column and for recirculating a part of the distillate in the second distillation column back into the first distillation column,
a discharge path for discharging the remaining part of the distillate in the second distillation column from the discharge port of the distillate in the second distillation column into the outside of the system, and
a means for adjusting the reflux ratio between the reflux pathway and the discharge path.

5. The system for purifying tetrahydrofuran according to claim 3, wherein the means for adjusting the reflux ratio is a means for adjusting the reflux ratio between the reflux pathway and the discharge path in the range of 5:1 to 20:1.

6. The system for purifying tetrahydrofuran according to claim 3, further comprising a hydrogenation column in the course of a flow path for connecting a discharge port of a distillate in a first distillation column and a raw material supply port in a second distillation column.

7. The system for purifying tetrahydrofuran according to claim 4, further comprising a hydrogenation column in the course of a flow path for connecting a discharge port of a distillate in a first distillation column and a raw material supply port in a second distillation column.
